(19) United States
(12) Patent Application Publication
(10) Pub. No.: US 2012/0123423 A1
(43) Pub. Date: May 17, 2012

(54) PATIENT-SPECIFIC INSTRUMENTS FOR TOTAL HIP ARTHROPLASTY

(75) Inventor: James Craig Fryman, New Paris, IN (US)

(73) Assignee: ZIMMER, INC., Warsaw, IN (US)

(21) Appl. No.: 13/282,844

(22) Filed: Oct. 27, 2011

Related U.S. Application Data
(60) Provisional application No. 61/412,588, filed on Nov. 11, 2010.

(51) Int. Cl. A61B 17/56

(52) U.S. Cl. 606/89

(57) ABSTRACT

Patient-specific instruments for preparing bones, such as a proximal femur and an acetabulum in a total hip arthroplasty, to receive respective orthopedic prostheses. The guides include a femoral resection guide and a bone canal preparation guide, each having a surface conforming to at least one of a metaphysis and a femoral neck of a femur. The femoral resection guide includes a cut referencing surface to guide a cutting instrument, and the bone canal preparation guide includes a guide aperture sized to guide a rasping instrument. An acetabular guide includes a surface conforming to an acetabulum and a guide aperture for guiding a surgical instrument such as a reaming instrument. The patient-specific, conforming surfaces of each of the guides may be designed based on patient-specific anatomical data obtained from the use of imaging technology.

### CROSS REFERENCE TO RELATED APPLICATIONS

This application claims the benefit under Title 35, U.S.C. §119(e) of U.S. Provisional Patent Application Ser. No. 61/412,588, entitled PATIENT-SPECIFIC INSTRUMENTS FOR TOTAL HIP ARTHROPLASTY, filed on Nov. 11, 2010, the entire disclosure of which is hereby expressly incorporated by reference herein.

### BACKGROUND

1. Field of the Invention

The present invention relates to instruments for total hip arthroplasty. More particularly, the present invention relates to patient-specific instruments for total hip arthroplasty.

2. Description of the Related Art

A total hip arthroplasty procedure may be performed to repair the diseased or damaged cartilage of a hip joint. In the procedure, a surgeon may use instruments to prepare the damaged joint for receiving an orthopedic prosthesis. For example, during a total hip arthroplasty procedure, the surgeon may ream the acetabulum of a patient to prepare a reamed area for receiving an acetabular cup prosthesis, and rasp the proximal femur to provide a rasped area for receiving a femoral prosthesis that includes a stem and head portion. The femoral stem portion includes a bone-engaging surface configured to be accepted into the rasped area of the proximal femur, and the femoral head portion includes an articulating surface that may be designed to articulate with the acetabular cup prosthesis seated within the acetabulum, for example.

### SUMMARY

The present invention provides patient-specific instruments for preparing bones, such as a proximal femur and an acetabulum in a total hip arthroplasty, to receive their respective orthopedic prostheses. In one embodiment, a femoral resection guide includes a surface conforming to at least one of a metaphysis and a femoral neck of a femur, and a cut referencing surface to guide a cutting instrument for resecting a first portion of the proximal femur. In another embodiment, a bone canal preparation guide includes a first surface conforming to an unresected portion of at least one of a metaphysis and a femoral neck, a second surface conforming to a resected portion of at least one of the metaphysis and the femoral neck, and a guide aperture sized to guide a rasping instrument for rasping a canal portion of the femur. In yet another embodiment, an acetabular guide includes a surface conforming to an acetabulum of a patient and a guide aperture for guiding a surgical instrument such as an acetabular reaming instrument for reaming the acetabulum or an inserter instrument for inserting an acetabular cup prosthesis into the acetabulum. The patient-specific, conforming surfaces of each of the above-referenced guides may be designed based on patient-specific anatomical data obtained from the use of imaging technology.

According to an embodiment of the present invention, a femoral resection guide for guiding a cutting instrument for preparing a femur to receive a prosthesis includes a body having a proximal surface and a distal surface, the distal surface being contoured to rest against and substantially conform to at least one of a femoral neck and a metaphysis of the femur, the body defining a cut referencing surface that is configured to guide the cutting instrument for resecting a first portion of the femur.

According to another embodiment of the present invention, a bone canal preparation guide for guiding a rasping instrument for preparing a particular patient's bone canal of a femur to receive a prosthesis includes a body having a proximal surface and a distal surface, the distal surface having a first portion contoured to rest against and substantially conform to an unresected portion of at least one of a femoral neck and a metaphysis of the femur and a second portion contoured to rest against and substantially conform to a resected portion of at least one of the femoral neck and the metaphysis, the body including a guide aperture extending through the body from said proximal surface to the distal surface, the guide aperture dimensioned to guide the rasping instrument for rasping a canal portion of the femur.

According to yet another embodiment of the present invention, an acetabular guide for guiding an acetabular surgical instrument for preparing an acetabulum of a patient to receive an acetabular cup prosthesis includes a body having a first surface and a second surface, the first surface being contoured to rest against and substantially conform to an acetabular rim of the patient, the body including a guide aperture extending through the body from the second surface to the first surface, the guide aperture dimensioned to guide the acetabular surgical instrument.

In one form thereof, the present invention provides a femoral resection guide for use in interfacing with a proximal femur to guide a cutting instrument for preparing the proximal femur to receive a prosthesis, the femoral resection guide including a substantially U-shaped body having a pair of arm portions, a proximal surface and a distal surface; the body dimensioned between the proximal surface and the distal surface to be substantially entirely disposed between a head of the proximal femur and a metaphysis of the proximal femur, and the arm portions dimensioned for receipt about the neck of the proximal femur when the guide is interfaced with the proximal femur; the distal surface contoured to rest against and substantially conform to at least one of the femoral neck and the metaphysis of the proximal femur, and the body including a cut referencing surface configured to guide the cutting instrument for resecting the head of the proximal femur.

In another form thereof, the present invention provides a femoral canal preparation guide for use in interfacing with a resected proximal femur and to guide an instrument for preparing the canal of the proximal femur to receive a prosthesis, the femoral canal preparation guide including a body including a proximal surface and a distal surface, the distal surface having a conforming portion contoured to rest against and substantially conform to an unresected portion of a metaphysis of the proximal femur around a resection of a neck of the proximal femur, the body including a guide aperture extending through the body from the proximal surface to the distal surface, the guide aperture dimensioned to guide an instrument for preparing a canal of the proximal femur.

In a further form thereof, the present invention provides a kit of patient-specific guides for use in preparing a proximal femur to receive a prosthesis, the kit including a femoral resection guide having a body including a proximal surface and a distal surface, the distal surface contoured to rest against and substantially conform to at least one of a femoral neck and a metaphysis of the proximal femur, and a cut referencing surface configured to guide a cutting instrument for resecting the head of the proximal femur; and a femoral canal preparation guide having a body including a proximal surface and a distal surface, the distal surface having a first portion contoured to rest against and substantially conform to an unresected portion of at least one of a femoral neck and a metaphysis of the proximal femur, the body including a guide aperture extending through the body from the proximal surface to the distal surface, the guide aperture dimensioned to guide an instrument for preparing a canal of the proximal femur.

### BRIEF DESCRIPTION OF THE DRAWINGS

The above-mentioned and other features and advantages of this invention, and the manner of attaining them, will become more apparent and the invention itself will be better understood by reference to the following description of embodiments of the invention taken in conjunction with the accompanying drawings, wherein:

FIG. 1 is an exploded perspective view of a femur and an exemplary femoral guide of the present invention;

FIG. 2 is a medial side view of the femur with the exemplary femoral guide of FIG. 1 seated about the femoral neck of the femur;

FIG. 3 is a perspective view of the femur with the exemplary femoral guide of FIG. 1 seated about the femoral neck, also showing a cutting instrument used to resect the femoral head from the femur;

FIG. 4 is an anterior view of the exemplary femoral guide of FIG. 1 seated about the femoral neck and having a straight resection plane;

FIG. 5 is an anterior view of another embodiment of an exemplary femoral guide of the present invention seated about the femoral neck and having a straight cut guide slot;

FIG. 6 is an anterior view of another embodiment of an exemplary femoral guide of the present invention seated about the femoral neck and having a proximal, angled resection plane;

FIG. 7 is an anterior view of another embodiment of an exemplary femoral guide of the present invention seated about the femoral neck and having an angled cut guide slot;

FIG. 8 is an exploded perspective view of a femur and another exemplary femoral guide for placement on a resected proximal femur and including a guide bore to guide an instrument;

FIG. 9 is an exploded perspective view of the guide of FIG. 8 placed on the resected proximal femur and an instrument to be guided via the guide;

FIG. 10 is a cross-sectional view taken along line 10-10 of FIG. 9 showing a thickness of the guide of FIG. 8;

FIG. 11 is a perspective view of another embodiment of an exemplary femoral guide of the present invention further including an arm to assist with guiding an instrument;

FIG. 12 is a perspective view of an embodiment of an exemplary acetabular guide of the present invention seated upon and partially surrounding an acetabular rim and including an arm for guiding an instrument;

FIG. 13 is a cross-sectional view taken along line 13-13 of FIG. 12 showing a portion of the guide of FIG. 12 having a through bore and a bottom portion conforming to the acetabular rim;

FIG. 14 is a cross-sectional view taken along line 14-14 of FIG. 12 showing a thickness of a portion of the guide of FIG. 12 and a bottom portion conforming to the acetabular rim;

FIG. 15 is a perspective view of another embodiment of an exemplary acetabular guide of the present invention seated upon and surrounding an acetabular rim and including an arm for guiding an instrument;

FIG. 16 is a perspective view of an acetabular reaming instrument guided by the arm of the guide of FIG. 15 that is seated on the acetabular rim; and

FIG. 17 is a flow chart of an exemplary method of the present invention.

Corresponding reference characters indicate corresponding parts throughout the several views. The exemplifications set out herein illustrate exemplary embodiments of the invention and such exemplifications are not to be construed as limiting the scope of the invention in any manner.

### DETAILED DESCRIPTION

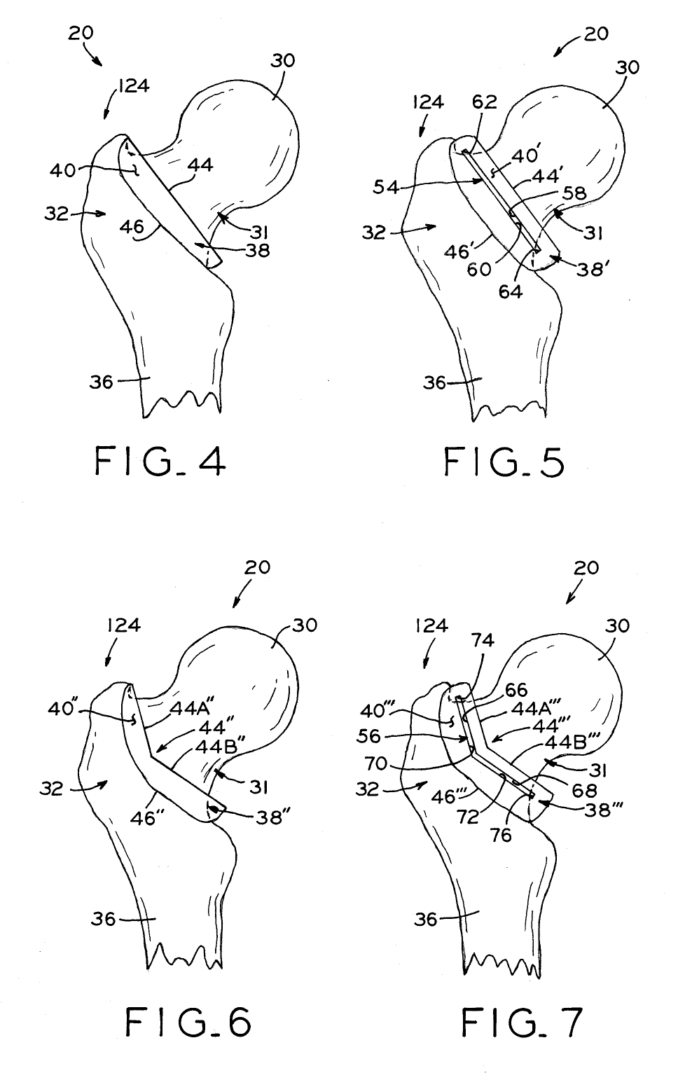

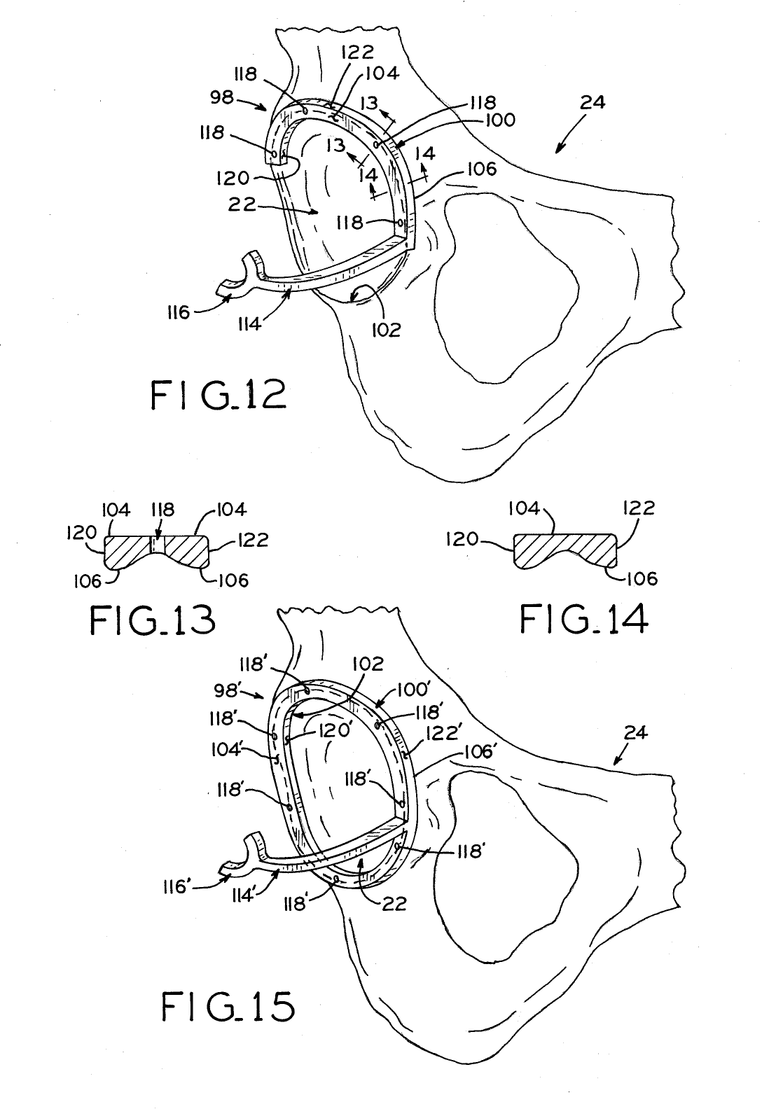

An orthopedic system of FIGS. 1-16 is provided for preparing femur 20 (FIG. 1) to receive a proximal femoral prosthesis (not shown) including a stem component and a head component and for preparing acetabulum 22 of pelvis 24 (FIG. 12) to receive acetabular cup prosthesis (not shown) in acetabulum 22 for articulation with the head component of the femoral prosthesis. Although the orthopedic system is described and depicted herein as being used to prepare femur 20 and acetabulum 22, the orthopedic system may be used to prepare other anatomical structures, such as the humerus, scapula, distal femur, tibia, radius, ulna, and other bones, to receive corresponding orthopedic prostheses.

In particular, the orthopedic system of the present invention is particularly suited for preparing the humerus and scapula bones due to the similarity in function and structures as compared to the proximal femur and acetabulum. In particular, the structure and function of the femoral resection guides, femoral canal preparation guides, and acetabular guides described below are closely analogous to the structure and function of corresponding humeral resection guides, humeral canal preparation guides, and scapular guides, respectively.

As shown in FIG. 1, the proximal end of femur 20 includes greater trochanter 26, lesser trochanter 28, head 30 extending from femoral neck 31, and metaphysis 32. Diaphysis 33 of femur 20 extends distally from metaphysis 32. Once femur 20 is prepared, a femoral prosthesis which includes appropriate offset and leg length dimensions is inserted into the prepared femur at an appropriate angle of anteversion.

As shown in FIG. 12, pelvis 24 includes acetabulum 22, a concave recess formed of three bones: the ischium (providing lower and side boundaries), illium (providing an upper boundary) and pubis (forming the midline) bones. An acetabular cup prosthesis is seated within a prepared acetabulum at appropriate angles of abduction and anteversion, for example, 45 degrees and 20 degrees, respectively. An abduction angle is determined with respect to a longitudinal axis of a patient's body from anterior to posterior and along a medial-lateral plane. The anteversion angle is determined with respect to the medial-lateral plane in a direction determined by which hip is undergoing the operation. As described further below, images taken of the hip joint include landmark imaging that may assist to create patient-specific guides conforming to the landmarks. Once appropriately placed on and conforming to the landmarks that indicate positioning for the desired angles of anteversion and abduction, the guide assists to prepare the acetabulum for seating an acetabular cup implant in the desired orientation.

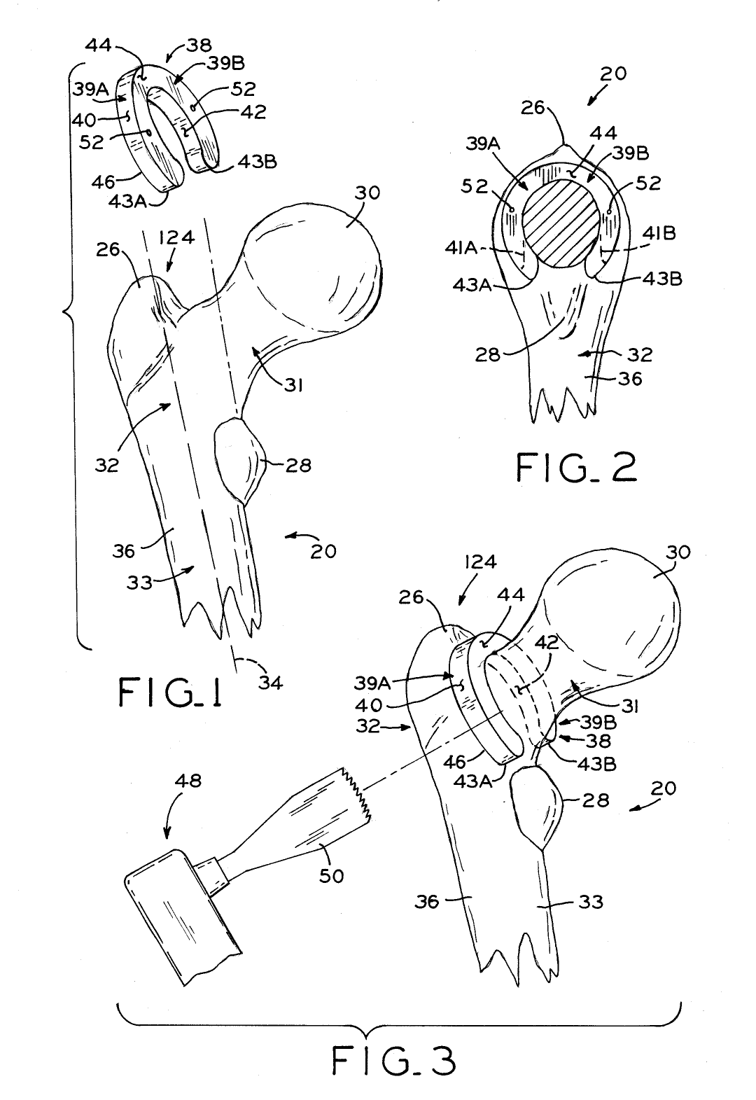

The exemplary guides of this disclosure, such as an exemplary femoral resection guide 38 described directly below, are designed in accordance with methods discussed further below. Referring to FIGS. 1-4, an exemplary first femoral resection guide 38 of the orthopedic system includes a substantially U-shaped component that seats against and conforms to landmarks on the body of the femur between the femoral head 30 and the greater trochanter 26. Referring to FIG. 1, femoral resection guide 38 includes a U-shaped component having arms portions 39A and 39B, an outer periphery 40, and an inner periphery 42. Peripheries 40 and 42 both connect a proximal cut guide surface 44 to a distal conforming surface 46, providing femoral resection guide 38 with a thickness. As may be seen in FIGS. 1-4 and as described below, due to the orientation of guide 38 when interfaced with the femur, proximal and distal surfaces 44 and 46 of guide 38 have medial and lateral aspects and could alternatively be referred to as proximal/medial and distal/lateral surfaces, respectively. Femoral resection guide 38 may additionally include holes 52 (FIGS. 1 and 2) for receipt of pins, for example, to temporarily secure guide 38 to femur 20.

In one embodiment, arm portions 39A and 39B may be rigid, having slightly curved ends 41A and 41B, respectively, as shown in dashed lines in FIG. 2. When arm portions 39A and 39B are rigid, respective ends 41A and 41B are disposed along respective vertical planes that are tangent to ends defining a greatest width of an outer profile of the neck against which inner periphery 42 of femoral resection guide 38 is seated. Thus, arm portions 39A and 39B do not significantly wrap around femoral neck 31 as arm portions 39A and 39B are made of a rigid material to be seated against an outer profile of neck 31. Alternatively, in another embodiment, arm portions 39A and 39B may be flexible, having curved ends 43A and 43B, respectively, as shown as solid lines in FIG. 2. When arm portions 39A and 39B are flexible, respective ends 43A and 43B are disposed within and between respective vertical planes that are tangent to ends defining a greatest width of an outer profile of the neck against which inner periphery 42 of femoral resection guide 38 is seated. Thus, in this embodiment, arm portions 39A and 39B significantly wrap around femoral neck 31, and arm portions 39A and 39B are made of a flexible material to be seated against an outer profile of neck 31.

In an embodiment, to reduce the amount of the acetabular capsule that needs to be resected, guide 39 may be half a U-shape (not shown) and have only a single arm portion (either arm portion 39A or arm portion 39B depending on the surgical approach) in order to reduce the amount of soft tissue clearance required to use the guide. The half U-shaped guide would only conform to the anterior or posterior geometry of the proximal femur, depending on the surgical approach. Either of the half U-shaped guides (arm portion 39A or arm portion 39B) may be additionally positioned with the use of temporary pins through the guide into femur 20. Similarly, each of the guides described herein may be temporarily secured into position with fasteners such as pins, screws, and like devices.

As may be seen in FIGS. 1-7, femoral resection guide 38 advantageously has a relatively small overall profile, which allows guide 38 to be interfaced with the proximal femur using minimized incisions with consequent minimized exposure of the proximal femur. In particular, the body of guide 38 is dimensioned between the proximal surface 52 and the distal surface 46 of guide 38 with a thickness such that guide 38 is substantially entirely disposed along the neck 31 of the femur and between head 30 of the proximal femur and the metaphysis 32 of the proximal femur.

Inner periphery 42 substantially conforms to, and is a negative of, the outer profile of femoral neck 31 and is designed based on patient-specific imaging data regarding the shape of the outer profile of femoral neck 31. Distal conforming surface 46 substantially conforms to and is a negative of a portion of at least one of metaphysis 32, femoral neck 31, greater trochanter 26, and lesser trochanter 28 of femur 20. Further, distal surface 46 and/or extensions from distal surface 46 may conform to patient-specific deformities and/or irregularities of femur 20. The proximal cut guide surface 44 is a substantially planar cut referencing surface, or cut guide plane, that guides an instrument, such as a reciprocating saw 48 (FIG. 3) along a resection path determined by the shape and angle of the cut guide plane. The resection, for example, may be planned along an exterior surface of the body of femoral resection guide 38, such as proximal surface 44, but may alternatively be positioned along another exterior or interior surface of the body. Blade 50 of reciprocating saw 48 resects femoral head 30 from femoral metaphysis 32 along the cut guide plane defined by proximal cutting surface 44. Alternatively, femoral resection guide 38 may facilitate the marking of a cut line to indicate an area for resection on or about the femoral head.

Referring to FIGS. 5-7, alternative embodiments of an exemplary femoral resection guide 38 are shown as femoral resection guides 38′ (FIG. 5), 38″ (FIGS. 6), and 38′″ (FIG. 7). Certain features of femoral resection guides 38′, 38″, and 38′″ are similar to the embodiment shown in FIGS. 1-4 and are designated with similar reference numbers. Femoral resection guide 38″ (FIG. 6) includes proximal cutting surface 44″ that includes a pair of cut guide surfaces 44A″ and 44B″ angled with respect to one another to guide an instrument, such as reciprocating saw 48 (FIG. 3), along proximal cutting surface 44″ to create an angled resection on the proximal femur.

FIGS. 5 and 7 illustrate alternative embodiments of femoral resection guide 38 as femoral resection guides 38′ and 38′″ that may additionally or alternatively include cut guide slots 54 and 56, respectively, to guide blade 50 of reciprocating saw 48. Femoral resection guide 38′ (FIG. 5) includes a straight, or linear, cut guide slot 54 and femoral resection guide 38′″ (FIG. 7) includes an angled, or two-part cut guide slot 56. Slot 54 (FIG. 5) includes internal proximal surface 58 opposite internal distal surface 60, surfaces 58 and 60 connected by first and second internal side surfaces 62 and 64. Slot 56 (FIG. 7) includes first and second internal proximal surfaces 66 and 68 angled with respect to one another and opposite first and second internal distal surfaces 70 and 72 similarly angled with respect to one another, first surfaces 66 and 70 connected by a first internal side surface 74 and second surfaces 68 and 72 connected by a second internal side surface 76.

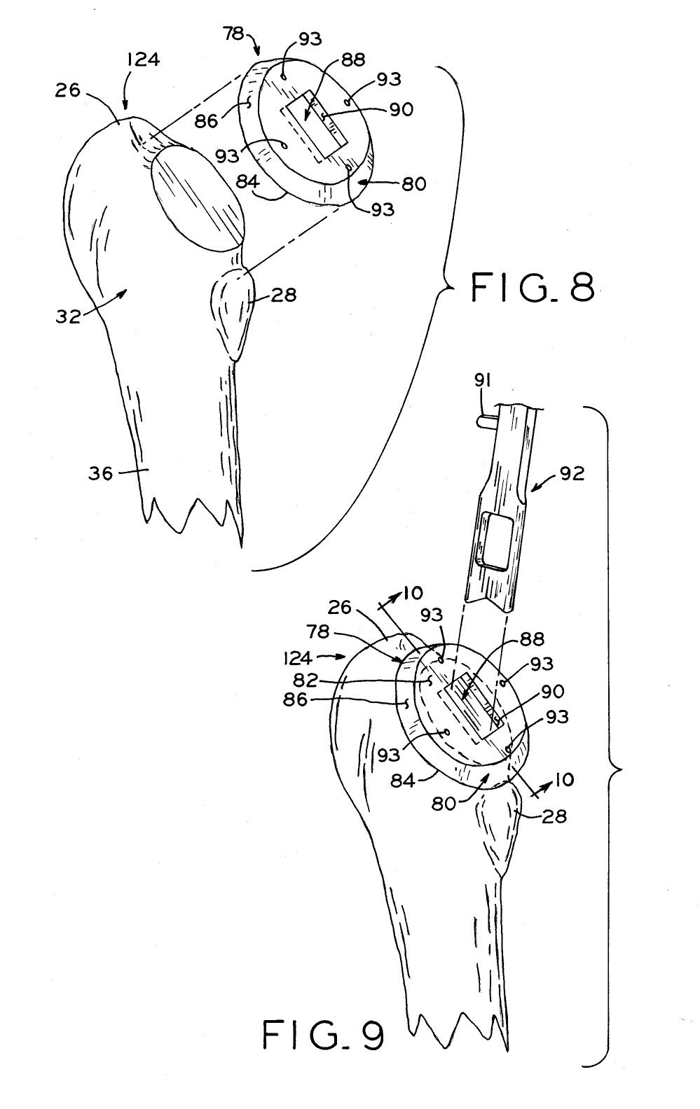

Referring to FIGS. 8 and 9, an exemplary femoral bone canal preparation guide 78 of an orthopedic system includes an annular base body 80 having a thickness between proximal surface 82 and distal surface 84, surfaces 82 and 84 connected by an outer peripheral wall 86, or outer periphery, of the base body. Base body 80 also includes central bore 88 defined by internal wall surfaces 90, or internal periphery, of the base body connecting proximal and distal surfaces 82 and 84. Internal wall surfaces 90 defining bore 88 may form, for example, a rectangular shape though other shapes are within the scope of the present disclosure. An instrument such as an end cutter, a reamer, or a rasp, for example, box osteotome 92 (FIG. 9), may be guidably received through and within bore 88 such that an outer periphery of the instrument will have a slight clearance with the inner periphery of the bore 88. As discussed below, reamers of progressively increasing diameter are utilized to ream the femoral canal after use of box osteotome 92 and are guidably received through bore 88 of bone canal preparation guide 78. Guide 78 may control both the angulation and the depth of instruments used for the rasping procedure.

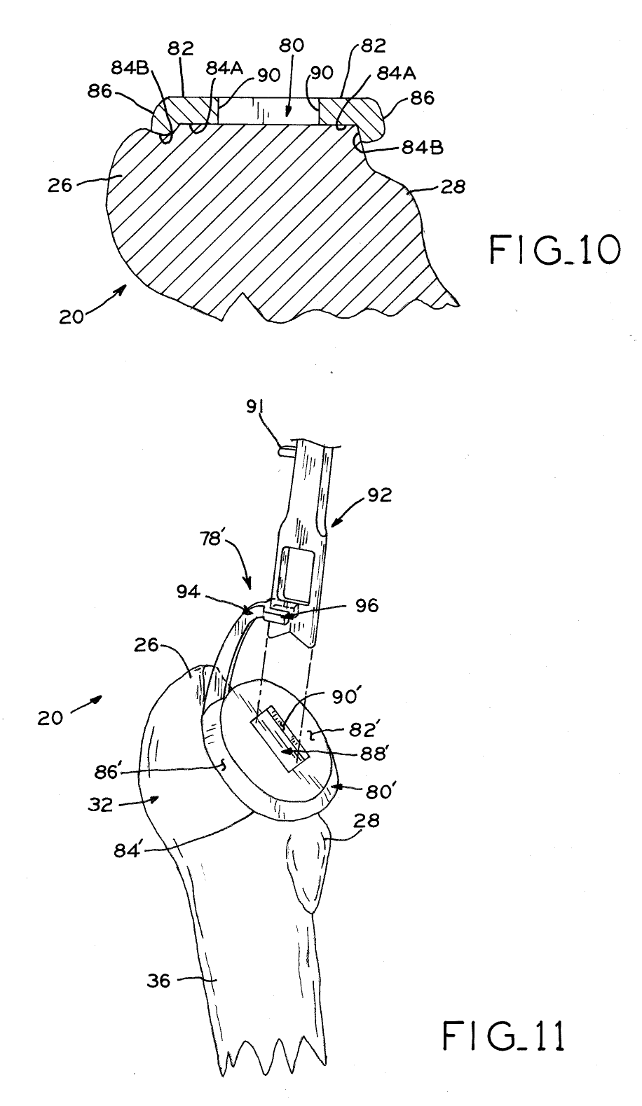

Referring to FIG. 10, a cross-sectional view of annular base body 80 and bore 88 of the guide 78 is shown disposed atop a resected proximal portion of femur 20 that is adjacent unresected portions of femur 20, particularly portions of at least one of metaphysis 32, femoral neck 31, greater trochanter 26, and lesser trochanter 28, such that distal surface 84A of bone canal preparation guide 78 conforms to the patient-specific shape of the resected portion and distal surface 84B conforms to the patient-specific shape of the unresected portions of femur 20. Further, distal surface 84B and/or extensions from distal surface 84B may conform to patient-specific deformities and/or irregularities along the unresected portions of femur 20. While the resected portions are typically planar, the resected portions may alternatively include non-planar surfaces that may be angled with respect to one another.

Femoral bone canal preparation guide 78 may additionally include holes 93 (FIGS. 8 and 9) for receipt of pins, for example, to temporarily secure guide 78 to femur 20.

Referring to FIG. 11, an alternate exemplary femoral bone canal preparation guide 78′ of the orthopedic system is similar to the embodiment shown in FIG. 8 and includes similar reference numbers. Bone canal preparation guide 78′, for example, includes annular base 80′, and further includes arm 94 extending proximally from base 80′. Arm 94 may be formed integrally with base 80′ and includes guide portion 96 attached at an arm end opposite an arm end attached to the annular base. Guide portion 96 has a sufficient length of engagement to mate with an instrument such as an end cutter, a reamer, or a rasp to guide the angular orientation of the received instrument. Further, guide portion 96 may be shaped as a half circle (FIG. 11) or a full circle (not shown), for which guide portion 96 may have a latching mechanism that opens the full circle to allow for mating with and mounting to an instrument. A full circle guide portion 96 may be disposed between two arm portions (not shown), each extending from base 80′ and connecting via full circle guide portion 96, which may include the latching mechanism to allow an instrument to be received into guide portion 96.

Arm 94 extends from the plane of the annular base to receive and guide an instrument such as an end cutter, a reamer, or a rasp within guide portion 96, for example. Arm 94 and guide portion 96 assist with guiding the received instrument along a desired, predetermined trajectory. Further, the received instrument may include a projection such as depth stop 91 (FIGS. 9 and 11) of box osteotome 92 that could interface with proximal surface 82 of guide 78 (FIG. 9) or with one of proximal surface 82′ and guide arm 94 of guide 78′ (FIG. 11) to set a desired depth stop. Similar to guide 78, guide 78′ may control both the angulation and the depth of instruments used for the rasping procedure.

As may be seen from FIGS. 8-11, annular base body 80 of femoral canal preparation guide 78 includes an outer periphery, such as along outer peripheral wall 86, extends just beyond the periphery of resection plane RP, though is dimensioned to be substantially co-extensive with the periphery of the resection plane RP (FIG. 8) of the metaphysis 32 of the proximal femur by which the femoral neck 31 is resected. In this manner, it may be seen that the conforming portion 84B (FIG. 10) of distal surface 84 of the guide 78 is annular in shape, and the overall dimensions of guide 78 are minimized such that there is minimal overhang of the outer periphery of the guide 78 with respect to the periphery of the resection plane RP.

Referring to FIG. 12, an exemplary acetabular guide 98 of an orthopedic system includes an annular base body 100 at least partially surrounding an acetabular rim 102 of acetabulum 22 and having a first, distal surface 106 conforming to rim 102 and a second, proximal surface 104, from which arm 114 proximally extends to receive and guide an instrument such as a reamer to ream the acetabulum or an instrument to seat an acetabular cup prosthesis. Distal surface 106 may additionally conform to bony structures (not shown) surrounding the acetabulum or include extension portions that wrap around bony structures of the acetabulum. Further, distal surface 106 and/or extensions from distal surface 106 may conform to patient-specific deformities and/or irregularities in rim 102 and the surrounding bony structures.

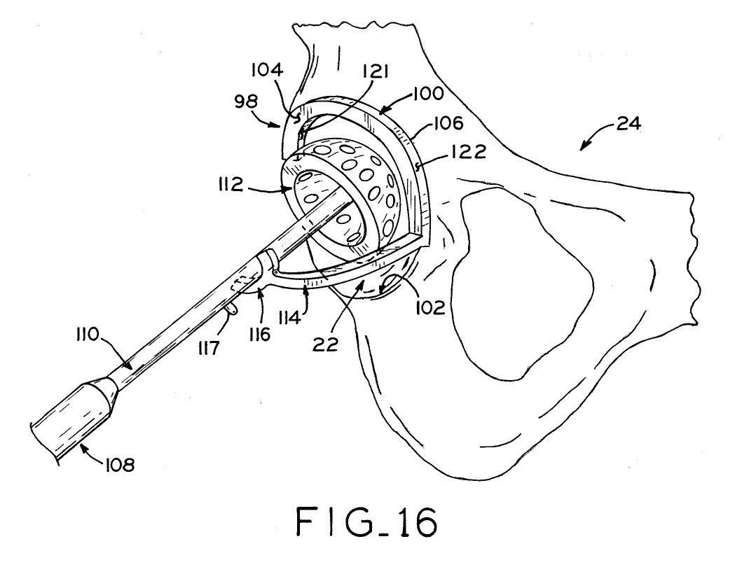

Surfaces 104 and 106 are connected by inner and outer walls 120 and 122, respectively. The reamer may be, for example, reamer 108 of FIG. 16 including inserter handle 110 connected at an end to reaming shell 112. Arm 114 has one end attached to annular base 100 and another end attached to guide portion 116. Guide portion 116 has a sufficient length of engagement to mate with a reaming or insertion instrument as described above to prevent angulation of the received instrument. Further, guide portion 116 may be shaped as a half circle (FIG. 12) or a full circle (not shown), for which guide portion 116 may have a latching mechanism that opens the full circle to allow for mating with and mounting to an instrument. A full circle guide portion 116 may be disposed between two arm portions (not shown), each extending from base 80′ and connecting via full circle guide portion 116, which may include the latching mechanism to allow an instrument to be received into guide portion 116.

Referring back to FIG. 12, guide portion 116 has a U-shaped design and may receive handle 110 of reamer 108, as shown in FIG. 16. Reamer 108 may include a projection such as depth stop 117 that could interface with one of proximal surface 104 and guide arm 114 of guide 98 to set a desired depth stop. Guide 98 may control both the angulation and the depth of the reaming instrument. Further, guide 98 may also guide the angulation of an acetabular cup inserter instrument (not shown) during insertion of the acetabular cup into the prepared acetabulum in a similar manner.

Referring back to FIG. 12, annular base body 100 may partially surround acetabular rim 102 and be connected to an end of arm 114 at an end of body 100. Internal wall surfaces may define holes for receipt of pins, for example, to temporarily secure guide 98 to acetabular rim 102. For example, hole 118 extends from proximal surface 104 to distal surface 106 of guide 98. FIG. 13 illustrates a cross-sectional view of hole 118 extending through the surfaces. Distal surface 106 conforms to the shape of the patient's bone. FIG. 14 shows a cross-sectional view of a portion of body 100 that has a thickness and does not include a bore. FIG. 15 illustrates an alternative embodiment of an exemplary acetabular guide in which annular base body 98′ substantially surrounds acetabular rim 102. Arm 114 may be positioned at any portion of the annular base body to set a desired trajectory for an instrument such as reamer 108 (FIG. 16).

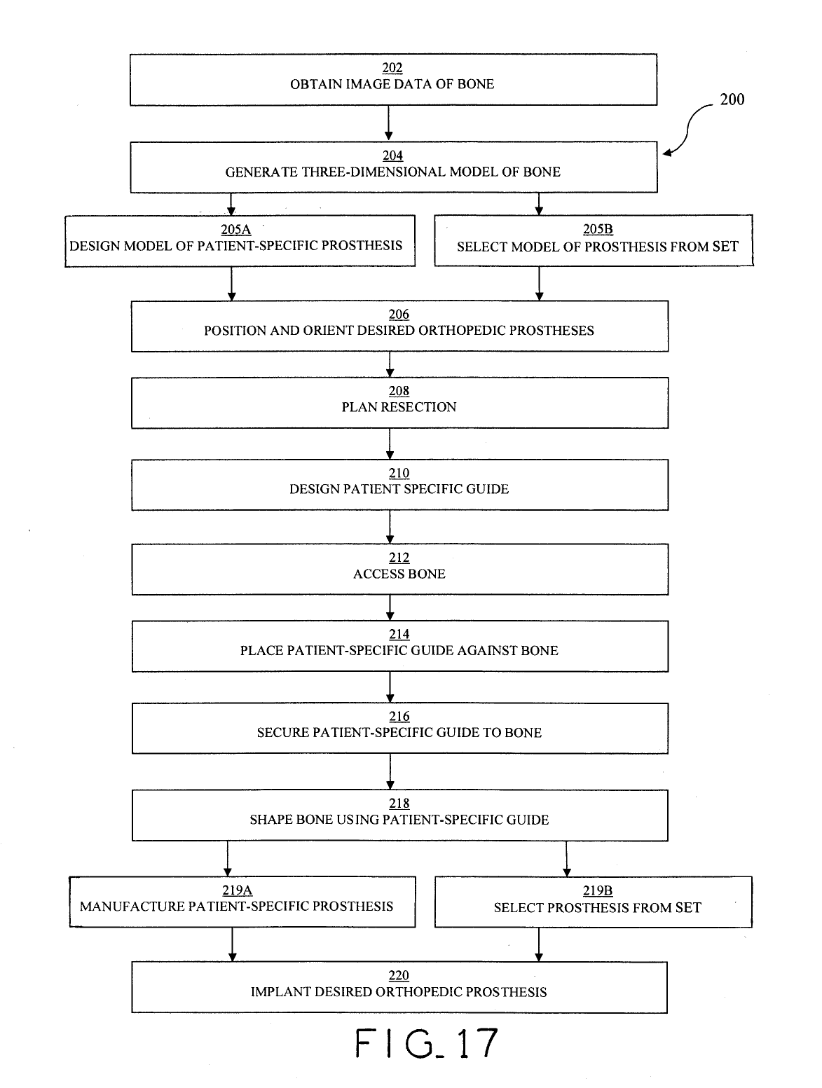

Referring to FIG. 17, an exemplary method 200 is provided for using one or more patient-specific guides such as guides 38-38′″ and/or 78-78′ to prepare proximal end 124 of femur 20 and a patient-specific guide, such as one of guides 98 or 98′, to prepare acetabulum 22 of pelvis 24.

First, in step 202 of method 200, the surgeon obtains image data of a patient's hip joint, including proximal end 124 of femur 20 and acetabulum 22 of pelvis 24, using a suitable imaging modality, such as magnetic resonance imaging (MRI), computed tomography (CT), ultrasound, or any another suitable imaging technique by which a volumetric, three dimensional image data set of the patient's joint may be obtained or calculated. For example, joint data may be obtained and manipulated as described in U.S. Pat. No. 5,768,134, issued Jun. 16, 1998, the entirety of which is hereby incorporated by reference herein. Additionally, images of the lower or distal leg (e.g., hip to foot) and the contralateral leg should also be acquired for the correct leg length calculation.

Optionally, the patient's hip joint may be placed in extension and/or tension prior to obtaining the imaging data. For example, in many patients who have arthritis or another disease or condition that affects the hip joint, it may be helpful for the surgeon to assess the joint space between the proximal end 124 of femur 20 and acetabulum 22 in tension to properly size the associated orthopedic prostheses and to optimally reconstruct the hip joint. A suitable brace (not shown) may be applied to pull on the ankle, for example, in order to place the hip joint in tension when the patient's leg is extended. In this manner, when the imaging data is obtained, femur 20, pelvis 24, and the surrounding soft tissue are all visible about the joint space such that the surgeon may evaluate soft tissue laxity to properly determine the size and position of the orthopedic prostheses, as discussed further below.

In addition to obtaining three dimensional imaging data of the hip joint when the hip is in extension, further imaging data may also be obtained of the hip joint in flexion, such as in mid flexion or to about 90° flexion. In one embodiment, additional three dimensional volumetric scans may be obtained in each of the foregoing positions. Alternatively, a two-dimensional imaging modality, such as an X-ray or fluoroscopy, may be used to obtain additional images in one or more positions in which the hip joint is in flexion, and a tension brace of the type described above may be used to assess laxity in the joint space. As described below, this additional imaging data may be used to construct a computer model of the hip joint and/or aid in the determination of the size and positioning of the orthopedic prostheses. For example and similarly as described above, joint data may be obtained and manipulated as described in U.S. Pat. No. 5,768,134, incorporated by reference above.

Next, in step 204 of method 200, the imaging data of femur 20 and pelvis 24 obtained during step 202 may be processed by a computer planning system which includes suitable computer software to generate a three-dimensional computer model of femur 20, pelvis 24, the lower leg, and the contralateral leg. For example, the computer planning system may include image processing software that is able to segment, or differentiate, the desired anatomic structure (e.g., bone tissue) from undesired structures (e.g., the surrounding soft tissue in the joint). Then, the image processing software generates a computer model of the desired structure. One suitable method for generating a computer model of a desired anatomic structure involves assigning a grey value to each pixel of the imaging data, setting a threshold grey value, and segmenting desired pixels from undesired pixels based on the threshold grey value. Another suitable method relies on using the density information gathered from the MRI or CT scans.

Using the computer model from step 204, the surgeon then selects a model of each desired prosthesis, for example, the desired femoral and acetabular cup prostheses. The femoral prosthesis may include modular components such as, for example, a neck, head and stem component. According to an exemplary embodiment of the present invention, the computer planning system displays the computer model to the surgeon so that the surgeon can evaluate the anatomy of the joint to determine the implant solution that is optimized for the anatomical needs of the patient. Selecting the model of each desired prosthesis may involve designing a custom, patient-specific prosthesis in step 205A of method 200 or choosing a standard prosthesis from a set of known orthopedic prostheses in step 205B of method 200. For example, in step 205A, the surgeon or computer planning system may design a model of a patient-specific implant that best matches the anatomical needs of the patient. Alternatively, in step 205B, the surgeon or computer planning system may access a digital database or library of known orthopedic prostheses, and select a model of a desired prosthesis from the database.

Additionally, leg length, offset, and angle of anteversion dimensions may be obtained from the patient-specific imaging data. With regard to the head component of a neck of a femoral prosthesis, the head component may be offset substantially horizontally, for instance, from a center of rotation of the head component oriented at an origin point that may or may not correspond to the original center of a femoral head prior to surgery, depending on the condition of the femoral head. The origin point does correspond, however, to a location at which a surgeon desires a center of the head component to be located and as determined via the patient-specific imaging data. The determined offset dimension may be measured and determined with reference to the longitudinal axis 34 of femoral stem 36 (FIG. 1). A longitudinal axis of the femoral head component (not shown) may be angled at an angle of anteversion that is angled with respect to the coronal (or medial/lateral) plane of the patient's body. The angle of anteversion is selected based off the patient-specific imaging data, which determines how the longitudinal axis of the femoral neck component is to be positioned along a plane that is set at or angled with respect to the medial/lateral plane at an angle ranging from, for example, 0 degrees to 15 degrees on average, though can be as high as 25 degrees.

Then, in step 206 of method 200, the surgeon uses the computer model of femur 20 and pelvis 24 to position and orient the desired orthopedic prosthesis for each region from step 205 relative to the bone. It is within the scope of the present invention that the orienting and positioning step 206 may occur after or simultaneously with the selecting step 205. According to an exemplary embodiment of the present invention, the surgeon overlays a digital representation or image of the desired prostheses onto the computer model of the associated bone to ensure the proper size of the desired prostheses and the proper orientation of the desired prostheses relative to the associated bone.

In certain embodiments, the surgeon or computer planning system may evaluate soft tissue laxity to properly size multiple prostheses simultaneously. For example, the computer planning system may evaluate soft tissue laxity in the hip joint to simultaneously size a proximal femoral prosthesis (not shown) and an acetabular cup prosthesis (not shown). Also, if multiple data sets of the hip joint in various positions of extension and flexion have been obtained, the same may be used for modeling a dynamic representation of the joint in which the surgeon may assess the joint in multiple positions of extension and flexion.

After the surgeon plans the size and location of the desired prostheses using the computer model during step 206, the computer planning system determines at step 208 of method 200 which portions of the bone must be removed from the computer model to receive the desired prostheses. In one embodiment, the computer planning system may identify for removal areas of overlap between the computer model of the bone and the digital model of the desired prostheses. For example, using the computer model of the bone and the digital model of the desired prostheses, the computer planning system may determine that a cavity must be further formed into the femoral canal laterally and posteriorly through a proximal-superior surface of femur 20, that a resection must be made of head 30 of femur 20 along a determined plane, for example, approximately 45 degrees to a long axis of femur 20, and that acetabulum 22 must be reamed to a desired depth in preparation to receive an acetabular cup prosthesis for articulation with a prosthetic head of femur 20.

Next, in step 210 of method 200, the computer planning system is used to design a custom, patient-specific guide, such as guide 98 or 98′ for acetabulum 22 and/or at least one of guide 38-38′″, 78, and 78′ for femur 20 based on the calculations from step 208. Each patient-specific guide may be an entirely custom product that is manufactured using a rapid prototyping process, such as 3-D printing, stereolithography, selective laser sintering, fused deposition modeling, laminated object manufacturing, or electron beam melting, for example. Alternatively, each patient-specific guide may be manufactured by removing material from a near net-shape blank or standard guide.

The patient-specific surgical guides may be provided in the form of a kit to the surgeon for use in surgery. For example, a kit including a custom, patient-specific femoral head resection guide, a custom, patient-specific femoral bone canal preparation guide, and a custom, patient-specific acetabular reaming and/or acetabular cup insertion guide may be packaged together and provided to the surgeon prior to surgery, each designed as described above based on patient-specific anatomical data.

Then, in step 212 of method 200, which corresponds to the beginning of the surgical procedure, the surgeon accesses at least one of acetabulum 22 and proximal end 124 of femur 20, such as via using a minimally invasive surgical procedure.

With an anterior approach, the hip joint is accessed anteriorly and, in particular, the surgeon accesses an interval between the sartorius muscle and tensor fascia latae. In this approach, the femoral resection guide 38 and the femoral bone preparation guide 78 may be fitted to the proximal femur as described herein without interfering with the iliofemoral ligament and the joint capsule attachment. Additionally, avoidance of the greater trochanter and the attachment of the obturator externus muscle using the anterior approach is also facilitated by the relatively small overall dimensions of the femoral resection guide 38 and the femoral bone preparation guide 78.

With a posterior approach, the hip joint is accessed posteriorly and, in particular, the surgeon may take the piriformis muscle and the short external rotators off the femur to access the acetabulum and femur while preserving the hip abductors. With this approach, the femoral resection guide 38 and femoral canal preparation guide 78 are dimensioned to avoid the lesser trochanter 28 and the hip joint capsule attachment. Additionally, avoidance of the greater trochanter and the attachment of the obturator externus muscle using the posterior approach is also facilitated by the relatively small overall dimensions of the femoral resection guide 38 and the femoral bone preparation guide 78.

Utilizing either the anterior or posterior approach, as shown in FIGS. 1-7 the femoral resection guide 38 is interfaced about neck 31 of the proximal femur and is dimensioned such that it is disposed, between its proximal and distal surfaces 52 and 46, entirely between femoral head 30 and metaphysis 32 of the femur.

Additionally, in either the anterior or posterior approach, the femoral canal preparation guide 78, as shown in FIGS. 8 and 9, is dimensioned such that its outer periphery is substantially co-extensive with the outer periphery of the resection by which the femoral head 30 is resected. This configuration minimizes overhang of the guide 78 while allowing conformity around the outer periphery of the guide 78 to the patient-specific contour of the metaphysis surrounding the resection plane.

Also, with either the anterior or posterior approach, the acetabular guide 98 is dimensioned to avoid the reflected head of the rectus femorus and the ischiofemoral ligament, and the base body 100 of the guide 98 is dimensioned to substantially conform to the overall dimensions of the acetabulum to minimize any overhang of the guide 98 over the acetabular rim so as to minimize any disruption of the joint capsule.

After the acetabulum 22 and proximal end 124 of femur 20 are exposed in step 212, the surgeon continues to step 214 of method 200 and places the respective acetabular or femoral patient-specific guide against acetabular rim 102 or femur 20. First, the surgeon orients the respective acetabular or femoral patient-specific guide with a distal surface of the guide facing toward acetabulum 22 or femur 20 and a proximal surface of the guide facing away from acetabulum 22 or femur 20, respectively, as shown in FIGS. 3 and 12.

According to an exemplary embodiment of the present invention, the femoral patient-specific guide conforms to femur 20 at predetermined locations. For example, a distal surface of the guide may be shaped to match the contour of femur 20 at respective predetermined locations of a proximal portion of femur 20. According to another exemplary embodiment of the present invention, the acetabular patient-specific guide conforms to acetabulum 22 at predetermined locations, such as at predetermined locations of the acetabular rim 102. For example, a distal surface of the acetabular guide may be shaped to match the contour of rim 102 at respective predetermined locations.

Once the acetabular or femoral guide is properly aligned with acetabular rim 102 or femur 20, respectively, the surgeon may temporarily secure the respective guide to acetabular rim 102 or femur 20 in step 216 of method 200. For example, the surgeon may temporarily secure femoral guide 78 to femur 20 by inserting screws, pins, or other suitable anchors (not shown) through apertures 93 in guide 78 and into the bone of femur 20. Any suitable number and arrangement of apertures may be provided in femoral guide 78. The acetabular guide, such as guide 98, may similarly be secured to the acetabular rim.

Next, in step 218 of method 200, the surgeon uses the femoral patient-specific guide to resect and prepare proximal end 124 of femur 20 or uses the acetabular patient-specific guide (discussed further below) to prepare acetabulum 22 in the manner discussed above. With respect to the femoral guide, during the procedure, at least one of femoral guide 38-38′″ (FIGS. 1-7) provides a resection plane or cut guide slot to guide a saw that resects the femoral head from the body along a predetermined angle and position. Further in operation, at least one of femoral guide 78-78′ (FIGS. 8-11) controls the position of a rasping instrument such as box osteotome 92 (FIGS. 9 and 11) relative to femur 20 so that the rasping instrument removes a desired portion of cancellous bone from femur 20. Thus, the depth of insertion of a rasp such as box osteotome 92 into femur 20 is governed by the distance between, for example, proximal surface 82 of guide 78 and femur 20. Additionally, box osteotome 92 may include depth stop 91 to interface with one of proximal surface 82 or arm 94 of guide 78.

The surgeon may use guide 38, for example, and reciprocating saw 48 to cut portions of femur 20. For example, the surgeon may use saw blade 50 of reciprocating saw 48 (FIG. 3) along referencing proximal surface 44 of guide 38 and a proximal surface of femur 20 to resect femoral head 30 from femoral metaphysis 32 along a predetermined cut line. It is also within the scope of the present invention that guide 38 may be provided with other cut referencing surfaces or cut slots so that the surgeon is able to cut other surfaces of femur 20 at predetermined locations.

In addition to resecting femur 20 using femoral resection guide 38, the surgeon may use femoral bone canal preparation guide 78 secured to femur 20, as shown in FIG. 8. For example, the surgeon first prepares the femoral canal with, for example, box osteotome 92 to receive a rasp. Reamers of progressively increasing diameter are utilized to ream the femoral canal. After a final reamer is removed, a rasp is inserted into the femoral canal. Progressively larger rasps are inserted to achieve optimal fit for the prosthesis. After a final rasp is used, the prosthesis is seated within the prepared femoral canal.

An exemplary femoral guide may include other features for preparing femur 20 to receive a proximal femoral prosthesis. For example, it is within the scope of the present invention that femoral resection guide 38 may include holes for drilling anchor holes into femur 20.

Additionally, acetabular guide 98 or 98′ may include similar other features for preparing the acetabulum to seat and receive an acetabular cup prosthesis. With respect to the acetabular guide, an acetabulum may receive a reamer shell such that, when force is applied to a handle connected to the reamer shell, the acetabulum is reamed to a predetermined depth at which point cartilage has been removed from the acetabulum, bone has been cut out to the periphery of the acetabulum, and a hemispheric shape of the acetabulum has been produced. Further, reaming provides a predetermined trajectory (incorporating desired angles of abduction and anteversion) for and prepares the acetabulum to receive an acetabular cup prosthesis.

The femoral and acetabular guides described throughout may be modified to include additional structures such as pin placer holes, drill guides, linked cut guides, and adjustable cut or drill guides, for example. Also, the femoral and acetabular guides may include navigation, orientation, and/or position sensor devices to allow modification of the guides themselves and/or to allow adjustability of the guides during use.

After preparing acetabulum 22 or femur 20 in step 218, the desired prostheses are implanted. Providing the desired prostheses may involve manufacturing custom, patient-specific prostheses in step 219A of method 200 based on the patient-specific prostheses designed during step 205A. Alternatively, providing the desired prostheses may involve choosing standard prostheses from a set of known orthopedic prostheses in step 219B of method 200 based on the model selected during step 205B.

According to an exemplary embodiment of the present invention, a patient-specific proximal femoral prosthesis may be provided in step 219A that is sized and shaped to replicate the portion of bone that was removed from femur 20 using femoral resection guide 38. However, if the natural articulating surface of femur 20 had been damaged or had deteriorated, the patient-specific proximal femoral prosthesis may be sized and shaped to replicate the portion of bone that was removed from femur 20 using femoral resection guide 38, as well as the portion of bone that was missing from femur 20 due to disease or traumatic injury, for example. In this embodiment, an articulating surface of the proximal femoral prosthesis may be sized and shaped to replicate the natural articulating surface of the femoral head of femur 20. According to another exemplary embodiment of the present invention, a patient-specific acetabular cup prosthesis may be similarly provided in step 219A to be sized and shaped to replicate the portion of bone removed from the acetabulum 22 using guide 98, for example, and to have an articulating surface for articulation with the articulating surface of the proximal femoral prosthesis.

Finally, in step 220 of method 200, the surgeon implants the desired proximal femoral prosthesis in proximal end 124 of femur 20 and/or the desired acetabular cup prosthesis in acetabulum 22 of pelvis 24. An exemplary proximal femoral prosthesis includes a top articulating femoral head surface for articulation with an articulating or inner bearing surface of an acetabular cup prosthesis.

While this invention has been described as having exemplary designs, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A femoral resection guide for use in interfacing with a proximal femur to guide a cutting instrument for preparing the proximal femur to receive a prosthesis, said femoral resection guide comprising:
a substantially U-shaped body having a pair of arm portions, a proximal surface and a distal surface;
said body dimensioned between said proximal surface and said distal surface to be substantially entirely disposed between a head of the proximal femur and a metaphysis of the proximal femur, and said arm portions dimensioned for receipt about the neck of the proximal femur when said guide is interfaced with the proximal femur;
said distal surface contoured to rest against and substantially conform to at least one of the femoral neck and the metaphysis of the proximal femur, and
said body including a cut referencing surface configured to guide the cutting instrument for resecting the head of the proximal femur.

2. The femoral resection guide of claim 1, wherein said arm portions include interior surfaces which are contoured to rest against and substantially conform to the femoral neck of the proximal femur.

3. The femoral resection guide of claim 1, wherein said proximal surface defines said cut referencing surface, and said cut referencing surface is one of a linear surface and an angled surface.

4. The femoral resection guide of claim 1, wherein said cut referencing surface is formed as a slot in said body.

5. A femoral canal preparation guide for use in interfacing with a resected proximal femur and to guide an instrument for preparing the canal of the proximal femur to receive a prosthesis, said femoral canal preparation guide comprising:
a body including a proximal surface and a distal surface, said distal surface having a conforming portion contoured to rest against and substantially conform to an unresected portion of a metaphysis of the proximal femur around a resection of a neck of the proximal femur, said body including a guide aperture extending through said body from said proximal surface to said distal surface, said guide aperture dimensioned to guide an instrument for preparing a canal of the proximal femur.

6. The femoral canal preparation guide of claim 5, wherein said body includes a periphery, said periphery dimensioned substantially co-extensive with a periphery of the resection of the neck of the proximal femur.

7. The femoral canal preparation guide of claim 5, wherein said conforming portion of said body is substantially annular in shape.

8. The femoral canal preparation guide of claim 5, wherein said femoral canal preparation guide further includes a guide arm extending proximally from said body of said femoral canal preparation guide, said guide arm including a guide portion for guiding the instrument.

9. A kit of patient-specific guides for use in preparing a proximal femur to receive a prosthesis, said kit comprising:
a femoral resection guide having a body including a proximal surface and a distal surface, said distal surface contoured to rest against and substantially conform to at least one of a femoral neck and a metaphysis of the proximal femur, and a cut referencing surface configured to guide a cutting instrument for resecting the head of the proximal femur; and
a femoral canal preparation guide having a body including a proximal surface and a distal surface, said distal surface having a first portion contoured to rest against and substantially conform to an unresected portion of at least one of a femoral neck and a metaphysis of the proximal femur, said body including a guide aperture extending through said body from said proximal surface to said distal surface, said guide aperture dimensioned to guide an instrument for preparing a canal of the proximal femur.

10. The kit of claim 9, wherein said proximal surface of said femoral resection guide defines said cut referencing surface, and said cut referencing surface is one of a linear surface and an angled surface.

11. The kit of claim 9, wherein said body of said femoral resection guide is dimensioned between said proximal surface and said distal surface thereof to be substantially entirely disposed between a head of the proximal femur and a metaphysis of the proximal femur when said femoral resection guide is interfaced with the proximal femur.

12. The kit of claim 9, wherein said body of said femoral resection guide is substantially U-shaped, and includes a pair of arm portions dimensioned for receipt about the femoral neck of the proximal femur.

13. The kit of claim 12, wherein said arm portions include interior surfaces which are contoured to rest against and substantially conform to the femoral neck of the proximal femur.

14. The kit of claim 9, wherein said body includes a periphery, said periphery dimensioned substantially co-extensive with a periphery of a resection of the neck of the proximal femur.

15. The kit of claim 9, wherein said femoral canal preparation guide further includes a guide arm extending proximally from said body of said femoral canal preparation guide, said guide arm including a guide portion for guiding the instrument.
